(19) United States
(12) Patent Application Publication
(10) Pub. No.: US 2002/0082564 A1
(43) Pub. Date: Jun. 27, 2002

(54) Camouflaged syringe arrangement

(76) Inventor: Tuan Pham, Milwaukee, WI (US)

Correspondence Address: William L. Falk, Andrus, Sceales, Starke & Sawall, LLP, Suite 1100, 100 East Wisconsin Avenue, Milwaukee, WI, 53202-4178, US

(21) Appl. No.: 09/749,145

(22) Filed: Dec. 27, 2000

(51) Int. Cl. A61M 5/32

(52) U.S. Cl. 604/192

(57) ABSTRACT

A syringe arrangement has a syringe holder for holding the syringe cartridge in which fluid medication is stored and a needle coupled to the syringe cartridge to communicate fluidically with the syringe cartridge. A plunger is slidably mounted with respect to the syringe holder for engagement with the syringe cartridge to expel the medication from the syringe cartridge through the needle for administering an injection to a patient. The syringe holder and the plunger take the form of a toy which is adapted to be handled by a patient without the syringe cartridge and the needle, and which camouflages the syringe cartridge and the needle in the syringe holder immediately prior to the administration of an injection to the patient.

### FIELD OF THE INVENTION

This invention relates broadly to medical equipment and, more particularly, pertains to a concealed and disguised syringe for delivering local anesthesia or medical substances to patients, especially children, with needle phobia.

### BACKGROUND OF THE INVENTION

The administration of medication to patients is extremely useful and often necessary during the course of many different medical and dental procedures. Medications can be dispensed in a variety of ways, such as orally, by inhalation, or by injection through a needle of a syringe. Injections are often the preferred means of delivery due to the short time lag between the injection of the medication and the resulting benefit to the patient. The benefit can be realized almost immediately because the medication can be injected directly to the desired area, or may be directly injected intravenously into the patient's blood stream. This can be differentiated with medication taken orally where there is usually a substantial lapse while the ingested medication is digested and delivered to the targeted site within the patient's body.

Although the injection of medication has various benefits and efficiencies, it is one of the least preferred methods for the patient to receive medication. In the administration of most injections, there is some measure of pain and discomfort caused by piercing of the skin by the needle and forcefully introducing medication into the tissue. As a result of being subjected to such painful and uncomfortable injections, many patients harbor a fear of, and apprehension toward, such injections. Some patients refuse to receive injections, while others simply postpone or altogether avoid seeking medical or dental care rather than confront the possibility of being subjected to an injection. In addition, many small children, or pedo-patients, have a much lower threshold of pain than adults. Thus, what might be considered merely an uncomfortable injection for an adult can be viewed as excruciatingly painful by a child, thus adding to the difficulty in effectively administering an injection.

These factors are compounded in the practice of dentistry where multiple applications may be required, many of which must be administered in locations that are difficult to access, such as the mandibular site. Studies have indicated that a great part of the patient's apprehension stems from the fear created when he or she observes the physician, reaching for and depressing the syringe plunger to inject the medication. As a consequence of the injection, the patient, particularly the pedo-patient, may become traumatized whenever he or she sees a syringe in advance of a needle strike. Such trauma may over time subject the patient to increased anxiety and a constant fear of all syringes and needles associated therewith.

Accordingly, it would be desirable to be able to deceptively mask and shield the needle of the syringe from the view of the patient, especially a child, so as to reduce the trauma and anxiety that will be experienced when the syringe is located at the injection site. That is, it would be desirable to provide an ornamental or toy-like syringe holder bearing an outward appearance which when presented to the pedo-patient would ease and hopefully accelerate the cooperation of the child with the dentist. It is likewise desirable to provide a syringe holder which is able to easily and quickly receive replacable medicinal fluid cartridges and needles, and permit the doctor to have control over the movement of the needle so as to avoid the possibility of an accidental needle strike which might lead to HIV or Hepatitis C transmission. Such a camouflaged syringe arrangement would be even more appealing to patients if it incorporated a further distraction feature such as pressurized air or water, music, lighting, or vibration.

The prior art is replete with many different syringe structures designed for various reasons. Some of these are pen-shaped or pencil grip holders constructed to improve the handling of the syringe during injection. Others have needle shields and retractable arrangements to protect the deliverer of the medicine dosage from being pricked after injection. However, there remains a need for a syringe construction having a much more tolerable appearance which will decrease the level of needle phobia and alleviate the amount of trauma developed by the patient in the administration of medicinal substances by injection.

### SUMMARY OF THE INVENTION

The present invention advantageously provides an ornamental or playful syringe holder for discretely hiding a syringe needle from view of a patient, particularly a child, to whom an injection is to be administered so as to lessen the anxiety to be experienced by the patient.

It is a general object of the present invention to provide an interactive, camouflaged syringe arrangement which will overcome or ameliorate needle phobia.

It is one object of the present invention to provide a camouflaged syringe arrangement having a syringe holder crafted in the form of a toy which will be attractive to a patient and will not be recognized as a standard syringe arrangement.

It is a further object of the present invention to provide a camouflaged syringe arrangement which provides a physician with control over movement of the needle.

It is an additional object of the present invention to provide a camouflaged syringe arrangement with a retractable, spring-biased needle.

It is also an object of the present invention to provide a camouflaged syringe arrangement having a replacable, anti-rolling syringe cartridge which is easily loaded into an ornamental syringe holder.

Yet another object of the present invention is to provide a camouflaged syringe arrangement having a distraction feature such as blown air or water, vibration, lighting, or music incorporated therein.

In one aspect of the invention, a camouflaged syringe arrangement includes a syringe cartridge containing a medicinal fluid, and a needle coupled to the syringe cartridge to communicate fluidically with the syringe cartridge. A syringe holder is provided for holding the syringe cartridge and the needle such that the needle is normally hidden in the syringe holder. A plunger is slidably mounted for movement into and out of the syringe holder, the plunger being engagable with the syringe cartridge to force the medicinal fluid from the syringe cartridge through the needle and push the needle out of the syringe holder for administering an injection to a patient. The syringe holder and the plunger cooperate together to define a toy adapted to be handled by the patient without the syringe cartridge and the needle installed in the syringe holder. The syringe holder includes structure for selectively delivering a stream of pressurized fluid to the patient. The syringe cartridge has a rectangular cross section such that when the syringe cartridge is placed on a flat surface, the syringe cartridge will not roll. The syringe holder includes a locking device for locking the plunger in various positions relative to the syringe holder. The syringe holder also includes a cavity for receiving the syringe cartridge, and a window for viewing the medicinal fluid in the syringe cartridge. The syringe holder has a forward end and a rearward end, the forward end being formed with an opening for receiving the needle and a chamber interposed between the opening and the cavity such that there is communication from the cavity through the chamber and the opening. The rearward end is formed with a channel in communication with the cavity for receiving the plunger. A spring is positioned in the chamber, the spring having one end engagable with an end wall in the chamber and the second end joined to a syringe cartridge stop engagable with the syringe cartridge. The structure for delivering a stream of pressurized fluid preferably includes a duct having one end adapted to be connected to a source of pressurized fluid and a second end forming a pair of outlets in the syringe holder. The rearward end of the syringe holder is provided with a finger rest having structure for receiving a coupling connected to the source of pressurized fluid. The plunger further includes a thumb rest. The syringe holder preferably takes the form of a fish body while the plunger preferably takes the form of a fish tail. The needle is preferably retractable.

In another aspect of the invention, there is contemplated a method of providing a camouflaged syringe arrangement for reducing needle phobia. The method includes the steps of supplying a syringe cartridge containing a medicinal fluid; supplying a needle to be coupled to the syringe cartridge; supplying a syringe holder for holding the syringe cartridge and the needle; supplying a plunger which is slidably mounted with respect to the syringe holder and engagable with the syringe cartridge to force the medicinal fluid from the syringe cartridge through the needle for administering an injection to a patient; designing the syringe holder and the plunger in the form of a plaything; and presenting the syringe holder and plunger without the syringe cartridge and the needle to the patient for his or her amusement in advance of administering the injection.

In another aspect of the invention, a syringe arrangement has a syringe holder for holding a syringe cartridge in which fluid medication is stored and a needle is coupled to the syringe cartridge to communicate fluidically with the syringe cartridge. A plunger is slidably mounted with respect to the syringe holder for engagement with the syringe cartridge to expel the medication from the syringe cartridge through the needle for administering an injection to the patient. The syringe arrangement is improved wherein the syringe holder and the plunger take the form of a toy which is adapted to be handled by a patient without the syringe cartridge and the needle, and which camouflages the syringe cartridge and the needle in the syringe holder immediately prior to the administration of an injection to the patient.

Various other features, objects and advantages of the invention will be made apparent from the following description taken together with the drawings.

### BRIEF DESCRIPTION OF THE DRAWINGS

The drawings illustrate the best mode presently contemplated of carrying out the invention.

In the drawings:

FIG. 1 is a perspective view of a camouflaged syringe arrangement embodying the present invention;

FIG. 2 is a longitudinal, sectional view of the syringe arrangement of FIG. 1 showing the manner in which a syringe cartridge and a needle are loaded into a syringe holder;

FIG. 2A is a sectional view like FIG. 2 showing the syringe cartridge and the needle loaded into the syringe holder such that the needle is hidden in the nose of the syringe holder;

FIG. 3 is a sectional view like FIG. 2 without the syringe cartridge and the needle in a condition which may be presented to a patient;

FIG. 4 is a sectional view like FIG. 2 showing the syringe cartridge and the needle being urged forwardly by means of a plunger structure;

FIG. 5 is a sectional view like FIG. 4 showing the delivery of a medicinal fluid from the syringe cartridge through the needle for administration to the patient;

FIG. 6 is an enlarged perspective view of the needle and the syringe cartridge;

FIG. 7 is a cross sectional view taken on lines 7-7 of FIG. 3;

FIG. 8 is an enlarged cross sectional view taken on line 8-8 of FIG. 3.

### DETAILED DESCRIPTION OF THE INVENTION

Referring now to the drawings, and in particular FIG. 1, there is shown generally a camouflaged syringe arrangement 10 embodying the present invention. As will be described hereafter, the arrangement 10 exhibits a particularly designed outward or external appearance which will be readily attractive to a patient, particularly a young patient, about to receive a dose of local anesthesia or a fluid medicinal substance by injection in a doctor's or a dentist's office. It is the express purpose of the camouflaged syringe arrangement 10 to prevent or, at least markedly reduce, the needle phobia often experienced by the patient in advance of a needle strike. In the description to follow, it should be appreciated that the present invention is applicable for administering of an injection at any location in the body. However, the arrangement 10 is particularly useful in administering oral injections such as required in the practice of dentistry.

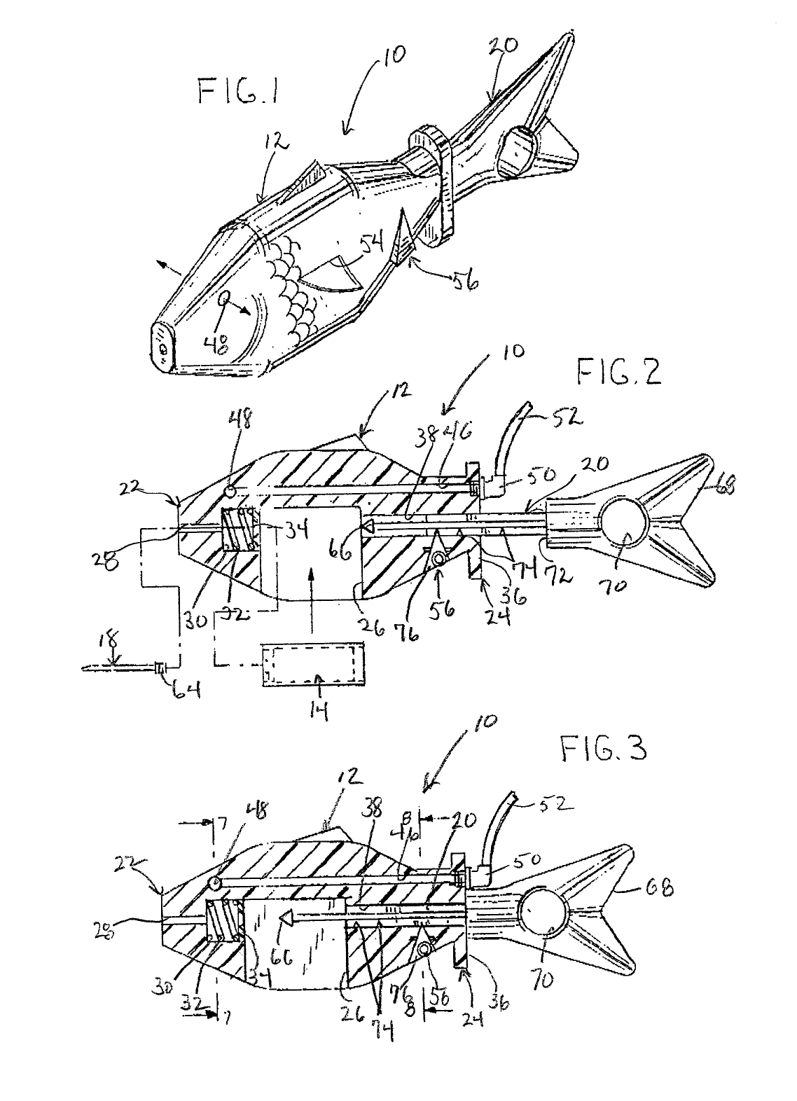

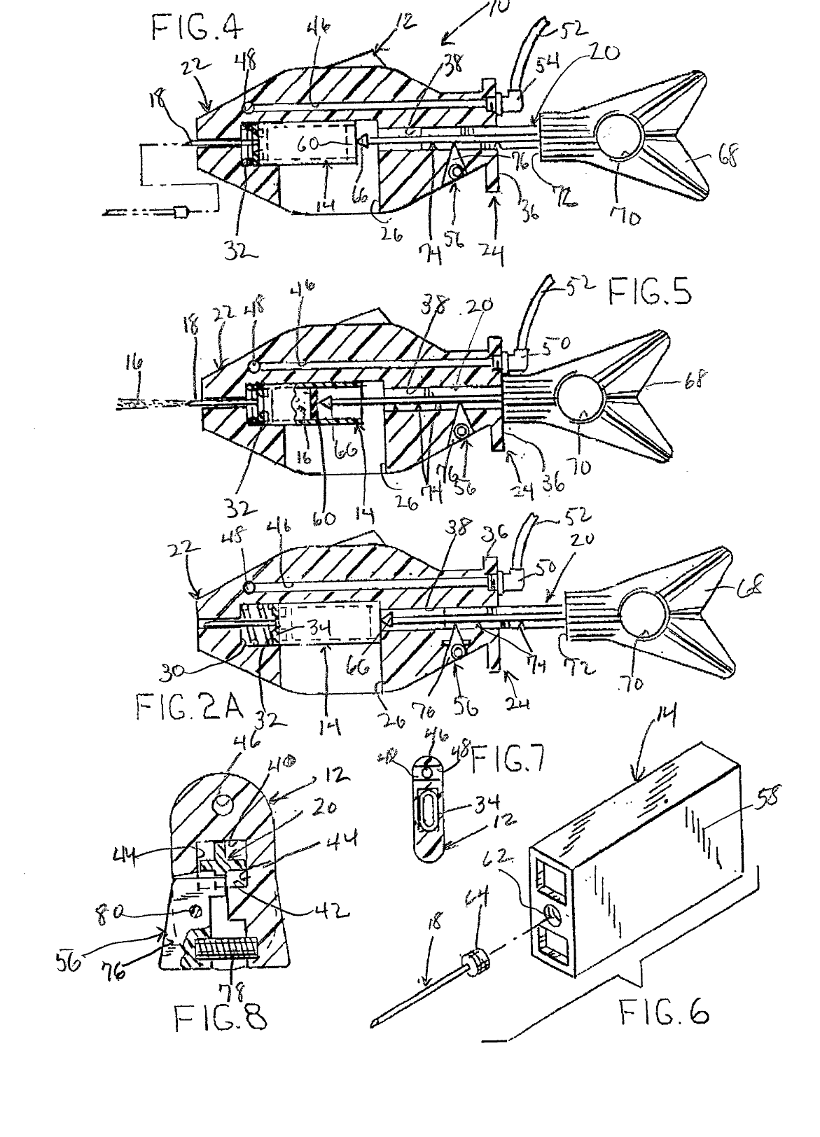

As seen in FIGS. 2, 2A, 4 and 5, the camouflaged syringe arrangement 10 is generally comprised of a syringe holder 12, a syringe cartridge 14 for holding a dose of medicinal fluid 16 (FIG. 5) therein, a needle 18 and a plunger 20 which is movable into and out of syringe holder 12.

Syringe holder 12 is preferably a one piece component having a forward end 22 and a rearward end 24 formed of solid material, such as plastic or the like. Opening into the central portion of the syringe holder 12 from the bottom thereof is an interior cavity 26 which is particularly sized and shaped to slidably receive the syringe cartridge 14 therein. Although not shown, the bottom of the syringe holder 12 may be equipped with a door or other retaining device to hold the syringe cartridge 14 in the cavity 26 if desired. Likewise, it should be understood that the cavity 26 may alternatively be formed from the side of the syringe holder 12. The forward end 22 of the syringe holder 12 has a horizontally disposed needle opening 28 which opens into an end wall of a chamber 30 adjacent to and in communication with the cavity 26. A coil spring 32 is positioned within the chamber 30 and has one end engaged within the end wall of chamber 30 and a second end attached to a syringe cartridge stop 34 which is engagable with the front end of cartridge 14. In FIG. 7, the syringe cartridge stop 34 is represented as an oval member owing to the shape of the syringe holder 12 at the location of the stop, but it can be appreciated that the stop 34 may be otherwise shaped. As will be appreciated, the coil spring 32 enables the needle 18 to be retractable. The rearward end 24 of the syringe holder 12 terminates in a flanged finger rest 36 and has a channel 38 which extends therethrough. The channel 38 is, for example, formed of a rectangular cross section having an upper wall 40, a lower wall 42 and sidewalls 44 (FIG. 6) which are engagable with the movable plunger 20. The channel 38 is in communication with the cavity 26 holding the syringe cartridge 14. An upper portion of the syringe holder 12 is designed with an elongated duct 46 which extends from the rearward end 24 of the holder 12 to the forward end 22 thereof. The forward end of the duct 46 has a pair of outlets 48 on each side of the syringe holder 12 as seen best in FIG. 1. The flanged finger rest 36 is suitably formed, such as by threading, to removably receive a coupling 50 having a tube or hose 52 connected with a source of pressurized fluid such as water or air which can be selectively delivered through the duct 46 and the outlets 48. At least one side of the syringe holder 12 is provided with a window 54 (FIG. 1) for observing the level of medicinal fluid 16 in the syringe cartridge 14 when the syringe cartridge 14 is installed in the cavity 26. The window 54 can also be used to check on fluid 16 during injection as well as aspiration. The syringe holder 12 is also furnished with a locking device 56 to be further described below for locking the plunger 20 in various axial positions relative to the holder 12.

Referring now to FIG. 6, the syringe cartridge 14, unlike conventional cylindrical cartridges, is expressly formed with a transparent housing 58 preferably having a rectangular cross section which will not roll away when placed on a flat surface such as a dental tray or table. In addition, the housing 58 is desirably shaped as shown so it can be easily but assuredly handled and loaded into the syringe holder 12. An inside, proximal end of the housing 58 includes the usual slidable rubber piston 60 (FIG. 5) to be urged by the plunger 20 therethrough in order to cause the fluid content 16 of syringe cartridge 14 to be expulsed during administration of the injection. A distal end of the housing 58 is provided with an internally threaded opening 62 for receiving a threaded hub 64 at the inner end of needle 18. It is to be understood that the syringe cartridge 14 will be prefilled with a medicinal fluid 16, and that the syringe cartridge 14 and the needle 18 are replacable.

Plunger 20 is slidably mounted for movement back and forth in the channel 38 of syringe holder 12. Plunger 20 has a crossed sectional construction having a forward end 66 which is spiked or pointed to piercingly engage the piston 60 forming the proximal end of the syringe cartridge 14. As seen in FIG. 8, the ends of the plunger 20 are designed to slidably engage the upper, lower and sidewalls, 40, 42, 44, respectively, of channel 38 in syringe holder 12. Plunger 20 also has an enlarged rearward end 68 having a circular thumb rest 70 and an inner surface 72 which is engagable and disengagable with the surrounding walls of the finger rest 36 at the rear of the syringe holder 12. As will be explained in greater detail below, the rearward end 68 of plunger 20 is contoured to compliment the shape of the syringe holder 12 for the purpose of making the syringe arrangement 10 patient friendly. Also, the bottom portion of the plunger 20 is provided with a series of spaced apart notches or detents 74 which are engagable with the apex of a triangular locking member 76 of the locking device 56 which is normally biased inwardly by a coil spring 78 best seen in FIG. 8. The locking member 76 is pivoted on a horizontally disposed pin 80 embedded in the syringe bolder 12. Applying finger pressure on the locking member 76 will rock the locking member about pivot pin 80 such that an upper portion of the locking member 76 will engage with one of the detents 74 to establish a particular linear axial position of the plunger 20. For example, FIGS. 2 and 2A show the plunger 20 in its rearwardmost position, while FIGS. 3 and 5 depict the plunger 20 in its forward most position with the enlarged rearward end 68 of plunger 20 flush in locked position against the finger rest 36.

In accordance with the invention, the syringe holder 12 and the plunger 20 are embodied in the form of a toy or plaything which is adapted to be handled by a patient, as presented by a doctor or dentist, without the syringe cartridge 14 and the needle 18 in place, and which camouflages the syringe cartridge 14 and the needle 18 in the syringe holder 12 immediately prior to the administration of an injection to the patient.

In the preferred embodiment, the syringe holder 12 and the plunger 20 are configured as a fish body and cooperating fish tail, respectively, which are typically constructed of a light weight, durable material such as plastic, which is appropriately colored to enhance the appearance thereof. The outlets 48 of the pressurized fluid duct 46 described above take the form of fish eyes on either side of the syringe holder 12. It should be noted that the representation of the syringe holder 12 and the plunger 20 as a fish is merely an example of the external appearance of the present invention. Other objects to be viewed by the patient as playthings such as animals, fruits, etc., could be used in lieu of the fish motif. The invention further contemplates that the toy or plaything may have an inanimate or animate form. The important principle is that the syringe holder 12 and the plunger 20 act not only to conceal and mask the syringe cartridge 14 and needle 18 from view immediately before an injection, but to quickly establish a friendly bonding between the patient and the doctor/dentist so as to lessen the anxiety felt by many patients. It is also helpful to incorporate a feature such as the duct 46 for delivering to the patients mouth, a stream of fluid which both serves to distract the patient and wash away any excess medicinal fluids spilled during the injection.

A child's anxiety usually stems from the new, unfamiliar surroundings and situations encountered in a dental office. Fortunately, most dentists are sensitive to this, and try to make their practice as welcoming as possible for patients with restful decor, colorful posters and children's books in its waiting rooms. It also helps to set a young patient's mind at ease by first chatting about other things and generally outlining what they can expect during the visit. In order to develop cooperation with the patient, the dentist presents the syringe arrangement 10 to the patient in the unloaded condition in the form shown in FIG. 3, that is without the syringe cartridge 14 and the needle 18. In this condition, the plunger 20 is fully advanced and is coupled together by the locking device 56 as one piece with the syringe holder 12. With the unloaded syringe arrangement 12 in the child's hands, the child has less tendency to become intimidated by the surrounding dental appliances. As such, it is intended that the child patient will become relaxed and easier to work with.

When it is necessary for the dentist to administer an injection, the dentist or his/her assistant can load the syringe cartridge 14 and the needle 18 out of the view of the patient. This is done by inserting the pre-filled syringe cartridge 14 into the cavity 26 until the top wall of the cartridge 14 abuts the top wall of the cavity 26 with the plunger 20 pulled back so that the pointed end 66 is located out of the cavity 26 as shown in FIG. 2. With one hand holding the cartridge 14 upwardly, the cartridge 14 is urged forwardly slightly into the chamber against the spring 32 so that the other hand can be used to screw the hub 64 of the needle 18 into the front end of the cartridge 14. It is envisioned that the needle 18 will be provided with a cover (not shown) to facilitate installation and keep the needle shaft sterile and out of direct contact with the hand. Once the needle 18 is fully screwed into the syringe cartridge 14, the cover is removed and the other hand holding the cartridge 14 is released so that the cartridge 14 will snap back against the rear wall of the cavity 26 and the needle 18 will be retracted or hidden in the needle opening 28 as shown in FIG. 2A.

At this point, the dentist with his/her first and second fingers engaged on finger rest 36 and his/her thumb engaged in the thumb rest 70 on plunger 20, applies an axial pushing force as represented in FIG. 4, such that the pointed end 66 of plunger 20 pushes the syringe cartridge 14 against the bias of spring 32 and against the syringe stop 34. With this motion, the cartridge 14 is pushed slightly into the chamber 30 until the spring 32 is fully compressed and the needle 18 begins to emerge from the needle opening 28. As the dentist continues to inject the syringe, the pointed end 66 of plunger 20 pushingly engages and captures the rubber piston 60 which is urged forwardly (FIG. 5) to expel the medicinal fluid 16 through the needle 18 and through the targeted site, in this case, the patient's mouth. During the injection, the dentist is able to visually inspect the amount of medicinal fluid being expelled through the needle by means of the window 54 in the side of the syringe holder 12. The dentist may also observe the amount of aspiration following the injection through the window 54. For this reason, it is important that the window 54 be aligned with the forward end of the syringe cartridge 14. Before, during and/or after the injection, the dentist may simultaneously apply pressurized water (or air) via the duct 46 and the outlets 48 to further distract the patient and purge away any spilled medicinal fluid 16 in the mouth. When the injection has been completed and the axial pushing force is terminated, the potential energy stored in spring 32 automatically returns the needle 18 to the hidden condition shown in FIG. 2A whereupon the patient experiences less emotional trauma as a result of not being able to see the injection taking place. The used needle 18 is unscrewed from the spent cartridge 14 using a cover or other protective extraction device and the cartridge 14 is removed from the cavity 26.

It should be appreciated that the syringe holder 12, syringe cartridge 14, needle 18 and plunger 20 may be purchased as a kit, or may be purchased as a retrofit system wherein the syringe holder 12 and plunger 20 are made to operate with a standard syringe cartridge and needle. Alternatively, a decorative toy-like or puppet-like cover could be used to camoflouge standard syringe arrangement. It should also be appreciated that the syringe holder 12 and plunger 20 are sized according to the particular application of the syringe. For instance, in the preferred embodiment, the width of the syringe holder 12 and the plunger 20 is minimized for maximum mobility in the mouth.

It is an advantage of the invention that the appearance, shape and interactive nature make the syringe arrangement 10 very unique and useful in medicinal practice.

The present invention thus provides a novel syringe arrangement 10 which delivers the necessary medicinal fluid by injection but discretely camouflages the syringe holder 12 and plunger 12 in a manner which will decrease anxiety of patients during insertion of a needle. This design is thus provided to reduce needle phobia symptoms and allow better cooperation between patients and physician.

While the invention has been described with reference to a preferred embodiment, those skilled in the art will appreciate that certain substitutions, alterations and omissions may be made without departing from the spirit thereof. For example, the invention broadly encompasses any medical instrument that is known to cause phobia to patients whether child or adult and effects the patient's behavior due to the appearance. Such medical instruments include, for example, scalpels, forceps, elevators, probes, drills, etc., all of which could be transformed to a more acceptable appearance while retaining their intended medical function. Accordingly, the foregoing description is meant to be exemplary only and should not be deemed limitative on the scope of the invention as set forth in the following claims.

I claim:

1. A camouflaged syringe arrangement comprising:
a syringe cartridge containing a medicinal fluid;
a needle coupled to the syringe cartridge to communicate fluidically with the syringe cartridge;
a syringe holder for holding the syringe cartridge and the needle such that the needle is normally hidden in the syringe holder, and
a plunger slidably mounted for movement into and out of the syringe holder, the plunger being engagable with the syringe cartridge to force the medicinal fluid from the syringe cartridge through the needle and push the needle out of the syringe holder for administering an injection to a patient,
the syringe holder and the plunger cooperating together to define a toy adapted to be handled by the patient without the syringe holder and the needle installed in the syringe holder.

2. The syringe arrangement of claim 1, wherein the syringe holder includes means for selectively delivering a stream of pressurized fluid to the patient.

3. The syringe arrangement of claim 1, wherein the syringe cartridge has a rectangular cross section such that when the syringe cartridge is placed on a flat surface, the syringe cartridge will not roll.

4. The syringe arrangement of claim 1, wherein the syringe holder includes a locking device for locking the plunger in various positions relative to the syringe holder.

5. The syringe holder of claim 1, wherein the syringe holder includes a cavity for receiving the syringe cartridge.

6. The syringe arrangement of claim 1, wherein the syringe holder includes a window for viewing the medicinal fluid in the syringe cartridge.

7. The syringe arrangement of claim 5, wherein the syringe holder has a forward end and a rearward end, the forward end being formed with an opening for receiving the needle and a chamber interposed between the opening and the cavity such that there is communication from the cavity through the chamber and the opening, the rearward end being formed with a channel in communication with the cavity for receiving the plunger.

8. The syringe arrangement of claim 7, including a spring positioned in the chamber, the spring having one end engagable with an end wall of a chamber and a second end joined to a syringe cartridge stop engagable with the syringe cartridge.

9. The syringe arrangement of claim 2, wherein the means for delivering a stream of pressurized fluid includes a duct having one end adapted to be connected to a source of pressurized fluid and a second end forming a pair of outlets in the syringe holder.

10. The syringe arrangement of claim 9, wherein the rearward end of the syringe holder is provided with a finger rest having structure for receiving a coupling connected to the source of pressurized fluid.

11. The syringe arrangement of claim 1, wherein the plunger includes a thumb rest.

12. The syringe arrangement of claim 1, wherein the syringe holder takes the form of a fish body.

13. The syringe arrangement of claim 1, wherein the plunger takes the form of a fish tail.

14. The syringe arrangement of claim 1, wherein the needle is retractable.

15. A method of providing a camouflaged syringe arrangement for reducing needle phobia, the method comprising the steps of:
supplying a syringe cartridge containing a medicinal fluid;
supplying a needle to be coupled to the syringe cartridge;
supplying a syringe holder for holding the syringe cartridge in the needle;
supplying a plunger which is slidably mounted with respect to the syringe holder and engagable with the syringe cartridge to force the medicinal fluid from the syringe cartridge through the needle for administering an injection to a patient;
designing the syringe holder and the plunger in the form of a plaything; and
presenting the syringe holder and plunger without the syringe cartridge and the needle to the patient for his or her amusement in advance of administering the injection.

16. In a syringe arrangement having a syringe holder for holding a syringe cartridge in which fluid medication is stored and a needle coupled to the syringe cartridge to communicate fluidically with the syringe cartridge, and a plunger slidably mounted with respect to the syringe holder for engagement with the syringe cartridge to expel the medication from the syringe cartridge through the needle for administering an injection to a patient, the improvement wherein:
the syringe holder and the plunger take the form of a toy which is adapted to be handled by a patient without the syringe cartridge and the needle, and which camouflages the syringe cartridge and the needle in the syringe holder immediately prior to the administration of an injection to the patient.
